(19) United States
(12) Patent Application Publication
(10) Pub. No.: US 2026/0041752 A1
(43) Pub. Date: Feb. 12, 2026

(54) PNEUMOCOCCAL CONJUGATE VACCINES WITH SAPONIN-BASED ADJUVANTS

(71) Applicant: NOVAVAX, INC., Gaithersburg, MD (US)

(72) Inventors: Nita PATEL, Gaithersburg, MD (US); Gale SMITH, Gaithersburg, MD (US); Rhonda FLORES, Gaithersburg, MD (US)

(21) Appl. No.: 19/293,773

(22) Filed: Aug. 7, 2025

Related U.S. Application Data
(60) Provisional application No. 63/680,542, filed on Aug. 7, 2024.

(51) Int. Cl. A61K 39/09; A61K 39/00; A61P 31/04

(52) U.S. Cl. CPC A61K 39/092; A61P 31/04; A61K 2039/55505; A61K 2039/55555; A61K 2039/55577; A61K 2039/55583; A61K 2039/60

(57) ABSTRACT

The invention is directed to an immunogenic composition comprising one or more conjugated bacterial capsular polysaccharides derived from Streptococcus pneumoniae serotypes, a saponin-based adjuvant, and a pharmaceutically acceptable carrier. The invention includes a method of preventing, treating or ameliorating a Streptococcus pneumoniae infection, disease or condition in a subject, comprising administering to the subject an effective amount of an immunogenic composition comprising one or more conjugated bacterial capsular polysaccharides derived from Streptococcus pneumoniae serotypes, a saponin-based adjuvant, and a pharmaceutically acceptable carrier.

### CROSS-REFERENCE TO RELATED APPLICATIONS

The present application claims priority to U.S. Provisional Application No. 63/680,542, filed Aug. 7, 2024, the entire contents of which is hereby incorporated by reference.

### FIELD OF THE DISCLOSURE

The present invention relates generally to immunogenic compositions comprising conjugated bacterial capsular polysaccharide antigens derived from serotypes of Streptococcus pneumoniae.

### BACKGROUND OF THE INVENTION

Streptococcus pneumoniae is a major pathogen causing pneumonia especially in young children and the elderly. To date, at least 98 different pneumococcal capsular serotypes have been identified. Pneumococcal infections include pneumonia, meningitis and febrile bacteremia; among the common non-invasive manifestations are otitis media, sinusitis and bronchitis. Pneumococcal conjugate vaccine helps to protect against bacteria that cause pneumococcal disease.

Pneumococcal vaccines are available to help prevent serious illness caused by the Streptococcus pneumoniae bacteria. These vaccines only cover some pneumococcal serotypes and are unable to protect against non-vaccine serotypes. There are at least ninety known serotypes, and specific serotypes causing disease vary by region, population, and are likely to mutate. Pneumococcal vaccines that are currently available for use in the United States include Prevnar-13®, Pneumovax 23®, Vaxneuvance™, Prevnar-20® and Capvaxive™. The current multivalent pneumococcal conjugate vaccines have been effective in reducing the incidence of pneumococcal disease associated with those serotypes present in the vaccines. Higher concentration of antibody titer is required to obtain opsonophagocytic killing responses.

There is a need to improve the immunogenic response produced by S. pneumoniae vaccines, and to identify and characterize emerging pneumococcal serotypes.

### SUMMARY OF THE INVENTION

The present invention relates generally to vaccines containing pneumococcal conjugates and saponin-based adjuvants such as Matrix-M. The vaccines are provided for administration through any suitable route, including intramuscular, subcutaneous and intranasal routes. The immunogenic compositions contain conjugated bacterial capsular polysaccharide antigens adjuvanted with a saponin-based adjuvant such as Matrix-M. Exemplary bacterial saccharide antigens are derived from serotypes of Streptococcus pneumoniae. The immunogenic compositions improve innate and adaptive immunity because of increased antigen density. The invention also provides methods for producing the immunogenic compositions and methods of stimulating immune responses with the compositions.

One embodiment of the invention is directed to an immunogenic composition comprising one or more conjugated bacterial capsular polysaccharides derived from Streptococcus pneumoniae serotypes, a saponin-based adjuvant, and a pharmaceutically acceptable carrier.

Another embodiment of the invention is directed to a method of preventing, treating or ameliorating a Streptococcus pneumoniae infection, disease or condition in a subject, comprising administering to the subject an effective amount of an immunogenic composition comprising one or more conjugated bacterial capsular polysaccharides derived from Streptococcus pneumoniae serotypes, a saponin-based adjuvant, and a pharmaceutically acceptable carrier.

In some aspects of the invention, the saponin-based adjuvant comprises nanoparticles from saponins extracted from Quillaja saponaria (soapbark) trees, cholesterol, and phospholipids.

In some aspects of the invention, the saponin-based adjuvant comprises fraction-A and fraction-C extract from Quillaja saponaria (soapbark) Molina.

In some aspects of the invention, the saponin-based adjuvant comprises Matrix-M adjuvant comprising Fraction A Matrix and Fraction C Matrix of Quillaja saponaria Molina extract.

In some aspects of the invention, the saponin-based adjuvant comprises Matrix-M comprising Fraction A Matrix and Fraction C matrix in a w/w ratio in the range 80:20 to 90:10. In some aspects of the invention, Fraction A Matrix and Fraction C Matrix are present in the saponin-based adjuvant in a w/w ratio of 85:15.

In some aspects of the invention, the saponin-based adjuvant is present at about 5 μg to about 100 μg, and may be present in an amount of 20 μg to 80 μg.

In some aspects of the invention, the immunogenic composition further comprises an aluminum adjuvant. In some aspects of the invention, the aluminum adjuvant is aluminum phosphate.

In some aspects, the conjugated bacterial capsular saccharides comprise saccharides of at least one, at least two, at least three, or all of S. pneumoniae serotype 3, serotype 6B, serotype 14 and serotype 23F.

In some aspects, the conjugated bacterial capsular polysaccharides are present in the immunogenic composition at about 0.5 μg to about 10 μg per serotype. In some aspects, the conjugated bacterial capsular saccharides are present at about 1 μg to about 5 μg per serotype.

In some aspects, the pharmaceutically acceptable carrier comprises succinate or L-Histidine.

In some aspects, the pharmaceutically acceptable carrier comprises an emulsifier. In some aspects, the emulsifier comprises at least one of polysorbate 20 (PS20), PS40, PS60, PS65, and PS80.

In some aspects, the composition is administered parenterally, transmucosally, transdermally, intramuscularly, intravenously, intradermally, intranasally, subcutaneously, or intraperitonealy. In some aspects, the composition is administered intramuscularly.

In some aspects, the composition is contained within a prefilled syringe.

### BRIEF DESCRIPTION OF THE DRAWINGS

The invention may be more completely understood in consideration of the following detailed description of various embodiments of the invention in connection with the accompanying drawings, in which:

FIG. 1 schematically illustrates the protocol timeline of a study comparing the immunological effect of Prevnar 13 compared to Prevnar 13 supplemented with Matrix-M adjuvant, as described in Example 1.

FIG. 2 shows results of an anti-Prevnar IgG titer assay for mouse blood serum after two injections and after three injections of a commercially available pneumococcal conjugate vaccine (Prevnar 13) that uses aluminum phosphate as the only adjuvant (solid squares) and the same vaccine augmented with the saponin-based adjuvant Matrix-M (open squares), in Example 1. The augmented vaccine shows an improved immunological response compared to the unaugmented vaccine after both two and three injections.

FIGS. 3A-3D show results of a multiplex opsonophagocytic assay (MOPA) on blood taken from mice at Day 42 in Example 1 for S. pneumoniae serotypes 3 (FIG. 3A), 6B (FIG. 3B), 14 (FIG. 3C) and 23F (FIG. 3D).

While the invention is amenable to various modifications and alternative forms, specifics thereof have been shown by way of example and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

The present invention is generally directed to pneumococcal conjugate vaccines that include saponin-based adjuvants, such as Matrix-M. The vaccines are provided for administration through any suitable route, including intramuscular, subcutaneous and intranasal routes. The immunogenic compositions contain conjugated bacterial capsular polysaccharide antigens adjuvanted with a saponin-based adjuvant such as Matrix-M. Exemplary bacterial saccharide antigens are derived from serotypes of Streptococcus pneumoniae. The immunogenic compositions improve innate and adaptive immunity because of increased antigen density. The invention also provides methods for producing the immunogenic compositions and methods of stimulating immune responses with the compositions

As used herein, and in the appended claims, the singular forms “a”, “an”, and “the” include plural referents unless the context clearly dictates otherwise. Thus, for example, reference to “a polysaccharide” can refer to one polysaccharide or to multiple such polysaccharides, and reference to “the method” includes reference to equivalent steps and/or methods known to those skilled in the art, and so forth.

As used herein, the term “consisting essentially” or “consists essentially” when referring to a composition indicates that components that are not otherwise identified as being part of the composition can be present in amounts that do not affect the safety or functionality of the composition. In some cases, such components can be present as common contaminants and/or additives of named components. In some cases, a composition can include less than 5% (e.g., less than 2%, less than 1%, or less than 0.1%) by weight of such components.

As used herein, the term “adjuvant” refers to a compound or substance that, when used in combination with an immunogen, augments or otherwise alters or modifies the immune response induced against the immunogen. Modification of the immune response may include intensification or broadening the specificity of either or both antibody and cellular immune responses.

As used herein, the terms “about” and “approximately,” when preceding a numerical value, indicates the value plus or minus 10% of the value. For example, “about 100” encompasses the range 90 and 110. When describing the limits of a range, the terms “about” and “approximately” should be understood to extend the limits of the range, i.e. extend the lower limit by 10% and the upper limit by 10%. Thus, for example, “from about 100 to about 200” should be read to cover the range from 90 to 220.

As used herein, the terms “immunogen,” “antigen,” and “epitope” refer to substances such as proteins, including glycoproteins, polysaccharides, conjugates, and peptides that are capable of eliciting an immune response.

As used herein, an “immunogenic composition” is a composition that comprises an antigen where administration of the composition to a subject results in the development in the subject of a humoral and/or a cellular immune response to the antigen. Immunogenic compositions of the present invention comprise conjugated bacterial capsular polysaccharide antigens, wherein the polysaccharide antigens are derived from serotypes of Streptococcus pneumoniae.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), microneutralization assay, multiplex opsonophagocytic assay (MOPA), and hemagglutination inhibition (HAI) titers.

As used herein, the term “vaccine” refers to an immunogenic composition, such as an immunogen derived from a pathogen, which is used to induce an immune response against the pathogen that provides protective immunity (e.g., immunity that protects a subject against infection with the pathogen and/or reduces the severity of the disease or condition caused by infection with the pathogen). The protective immune response may include formation of antibodies and/or a cell-mediated response. Depending on context, the term “vaccine” may also refer to a suspension or solution of an immunogen that is administered to a subject to produce protective immunity.

As used herein, the term “subject” includes humans and other animals. Typically, the subject is a human. For example, the subject may be an adult, a teenager, a child (2 years to 14 years of age), an infant (1 month to 24 months), or a neonate (up to 1 month). In some aspects, the adults are seniors about 65 years or older, or about 60 years or older. In typical aspects, the adults are about 60 to about less than 75 years old, or at least about 75 years old. In some aspects, the subject is a pregnant woman or a woman intending to become pregnant. In other aspects, subject is not a human; for example a non-human primate; for example, a baboon, a chimpanzee, a gorilla, or a macaque. In certain aspects, the subject may be a pet, such as a dog or cat.

As used herein, the term “pharmaceutically acceptable” means being approved by a regulatory agency of a U.S. Federal or a state or foreign government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

The present invention generally relates to immunogenic compositions comprising conjugated bacterial capsular polysaccharide antigens. The immunogenic compositions contain conjugated bacterial capsular polysaccharide antigens adjuvanted with a saponin-based adjuvant such as Matrix-M. Exemplary bacterial saccharide antigens are derived from serotypes of Streptococcus pneumoniae. The immunogenic compositions improve innate and adaptive immunity because of increased antigen density. The invention also provides methods for producing the immunogenic compositions and methods of stimulating immune responses with the compositions

The compositions stimulate immune responses against multiple S. pneumoniae serotypes. Advantageously, the immune responses may include broadly neutralizing antibodies directed to S. pneumoniae serotypes different from those used to prepare the compositions. In addition, the compositions exhibit excellent stability and may be stored for extended periods and may be produced in pre-filled syringes that are ready to administer. In some aspects, a pre-filled syringe (PFS) may already contain adjuvant and conjugated bacterial capsular saccharide antigens and may be stored for extended periods. Compositions disclosed herein need not be refrigerated (for example, at 2-8° C.), and show good stability at higher temperatures (e.g. room temperature, about 22-25° C.). The compositions disclosed herein therefore offer excellent convenience as well as excellent immune responses.

### Pneumococcal Conjugate Vaccines

Pneumococcal conjugate vaccine is a pneumococcal vaccine made with the conjugate vaccine method and used to protect infants, young children, and adults against disease caused by the bacterium Streptococcus pneumoniae (pneumococcus). It contains purified capsular polysaccharides of pneumococcal serotypes conjugated to a carrier protein to improve antibody response compared to the pneumococcal polysaccharide vaccine. Pneumococcal conjugate vaccine is used to protect infants, young children, and adults against disease caused by S. pneumoniae. It contains purified capsular polysaccharides of a number of pneumococcal serotypes conjugated to a carrier protein to improve antibody response compared to the pneumococcal polysaccharide vaccine.

Various pneumococcal conjugate vaccines have been approved for commercial use. For example, in the Prevnar family of vaccines, produced by Pfizer, the bacterial cell capsule sugars, a characteristic of these pathogens, are linked (conjugated) through reductive amination to CRM197, a nontoxic recombinant variant of diphtheria toxin. CRM197 is derived from the C7 strain of Cornebacterium diphtheriae grown in a medium of casamino acids and yeast extracts. Bacteria bearing the vaccine's polysaccharide sugars are grown separately in soy peptone broths. The resulting glycoconjugate produces a more robust immune response in most healthy persons. Aluminum, in the form of aluminum phosphate, is added to the commercial vaccine as an adjuvant to enhance the immune response.

The first version of Prevnar approved in the United States was the heptavalent Prevnar, PCV7, produced from the seven most prevalent strains of S. pneumoniae in the U.S. (serotypes 4, 6B, 9V, 14, 18C, 19F, and 23F). Prevnar PCV7 was approved for use in the U.S. in February 2000. The second version of Prevnar approved in the United States, in 2010, was Prevnar 13 (PCV13), which includes thirteen serotypes of pneumococcus (1, 3, 4, 5, 6A, 6B, 7F, 9V, 14, 18C, 19A, 19F, and 23F). Each 0.5 mL aqueous dose of the PCV13 vaccine is formulated to contain approximately 2.2 μg of each of Streptococcus pneumoniae serotypes 1, 3, 4, 5, 6A, 7F, 9V, 14, 18C, 19A, 19F, 23F saccharides, 4.4 μg of 6B saccharides, 34 μg CRM197 carrier protein, 100 μg polysorbate 80, 295 μg succinate buffer and 125 μg aluminum as aluminum phosphate adjuvant.

In April 2023, the FDA approved Prevnar 20 for the prevention of disease caused by the 20 different serotypes of S. pneumoniae contained in the vaccine (serotypes 1, 3, 4, 5, 6A, 6B, 7F, 8, 9V, 10A, 11A, 12F, 14, 15B, 18C, 19A, 19F, 22F, 23F, and 33F) for individuals 6 weeks through 17 years of age; and for the prevention of otitis media (ear infection) caused by 7 of the serotypes of Streptococcus pneumoniae contained in the vaccine for children 6 weeks through 5 years of age. Each 0.5 mL aqueous dose of the Prevnar 20 contains approximately 2.2 μg of each of S. pneumoniae serotypes 1, 3, 4, 5, 6A, 7F, 8, 9V, 10A, 11A, 12F, 14, 15B, 18C, 19A, 19F, 22F, 23F, 33F saccharides, 4.4 μg of 6B saccharides, 51 μg CRM197 carrier protein, 100 μg polysorbate 80, 295 μg succinate buffer, 4.4 mg sodium chloride, and 125 μg aluminum as aluminum phosphate adjuvant.

Capvaxive is a pneumococcal 21-valent conjugate vaccine (PCV21) manufactured by Merck and was approved for medical use in the United States in June 2024. It is indicated for the active immunization for the prevention of invasive disease caused by Streptococcus pneumoniae serotypes 3, 6A, 7F, 8, 9N, 10A, 11A, 12F, 15A, 15B, 15C, 16F, 17F, 19A, 20A, 22F, 23A, 23B, 24F, 31, 33F, and 35B in individuals 18 years of age and older; and the active immunization for the prevention of pneumonia caused by S. pneumoniae serotypes 3, 6A, 7F, 8, 9N, 10A, 11A, 12F, 15A, 15C, 16F, 17F, 19A, 20A, 22F, 23A, 23B, 24F, 31, 33F, and 35B in individuals 18 years of age and older. Each 0.5 mL aqueous dose contains a total of 84 μg of pneumococcal polysaccharide antigen (4 μg each of polysaccharide serotypes 3, 6A, 7F, 8, 9N, 10A, 11A, 12F, 15A, 15B (deOAc 15B), 16F, 17F, 19A, 20A, 22F, 23A, 23B, 24F, 31, 33F, and 35B) conjugated to approximately 65 μg of CRM197 carrier protein, 1.55 mg L-histidine, 0.50 mg of polysorbate (PS) 20, 4.49 mg sodium chloride, and water for injection.

Vaxneuvance is a 15-valent pneumococcal conjugate vaccine marked by Merck, and was approved for use in the United States in July 2021. It includes the same serotypes as PCV13, with the addition of serotypes 22F and 33F. These two serotypes were chosen because they have been found to become more prevalent in a population that has extensively used PCV13. Vaxneuvance is indicated for the active immunization for the prevention of invasive disease caused by Streptococcus pneumoniae serotypes 1, 3, 4, 5, 6A, 6B, 7F, 9V, 14, 18C, 19A, 19F, 22F, 23F and 33F in adults 18 years of age and older. Each 0.5 mL aqueous dose contains 2.0 μg each of S. pneumoniae polysaccharide serotypes 1, 3, 4, 5, 6A, 7F, 9V, 14, 18C, 19A, 19F, 22F, 23F, and 33F, and 4.0 μg of polysaccharide serotype 6B, 30 μg of CRM197 carrier protein, 1.55 mg L-histidine, 1 mg of polysorbate 20, 4.50 mg sodium chloride, and 125 μg of aluminum as aluminum phosphate adjuvant.

Embodiments of the invention are directed to the addition of a saponin-based adjuvant, such as Matrix adjuvant, to a pneumococcal conjugate vaccine, which results in an improved immunogenic response, as described in the examples below.

### Saponin-Based Adjuvants

Saponin-based adjuvants, i.e. adjuvants containing saponin, may be used in a pneumococcal conjugate vaccine. Saponins are glycosides extracted from the bark of the Quillaja saponaria Molina tree. These bark extracts contain a heterogeneous mixture of hundreds of related saponins with structurally different glycosylation or acylation patterns that can affect their biological activities.

Saponins are a large family of plant-derived glycoconjugates that share a triterpene structure with a variety of glycoside side chains. Saponins have traditionally been used for making soaps based on being amphipathic. Saponins also are used for making adjuvants based on having potent immune-stimulating properties, as taught, for example by Kensil et al., U.S. Pat. No. 5,057,540.

Quillaja saponaria Molina saponins can have a high degree of glycosyl O-acylation, a low degree of glycosyl O-acylation, or no glycosyl O-acylation in their naturally occurring forms. Saponins also can be chemically modified, for example by partial or complete deacylation or degradation. Saponins of Quillaja saponaria Molina in particular can have potent adjuvant activity, but also can be chemically unstable, show hemolytic activity, and be associated with immediate pain at injection sites. Saponin preparations based on defined compositions of purified saponin fractions of Quillaja saponaria Molina are described, for example, by Cox et al., PCT/AU1995/000670 (WO 96/11711).

Novavax's Matrix-M adjuvant is a formulation that is manufactured using two extracts of saponins of Quillaja saponaria Molina, termed saponin fraction A and saponin fraction C, which are described in detail below. To make Matrix-M adjuvant, saponin fraction A and saponin fraction C are separately mixed with cholesterol and phosphatidylcholine, in the presence of the detergent Mega-10 to form dispersions of approximately 40-50 nm-sized stable cage-like structures, designated Matrix-A and Matrix-C particles. The Mega-10 detergent is removed by diafiltration. The Matrix-A and Matrix-C particles are provided in formulations in phosphate buffer solution including 137 mM sodium chloride, 2.7 mM potassium chloride, and 9.8 mM phosphate, at pH 7.2. The Matrix-M adjuvant is obtained by mixing the Matrix-A and Matrix-C particles at a fixed weight ratio of 85:15 of Matrix-A particles to Matrix-C particles. It should be appreciated that the A (H5N1) HA glycoprotein associates with a Matrix-M component to make the H5-MNP, in other words the HA glycoprotein associates with a Matrix-A particle or a Matrix C particle of the Matrix-M adjuvant. Other ratios of Matrix-A and Matrix-C particles, for example in the ratio range of 80:20 w/w to 90:10 w/w, may provide improved immune response, depending on the antigen.

Considering saponins in more detail, as noted above, saponin preparations based on defined compositions of purified saponin fractions of Quillaja saponaria Molina are described, for example, by Cox et al., PCT/AU1995/000670 (WO 96/11711). Initially, formulation of saponins into particles was performed with a semi-purified, non-fractionated saponin extract from the bark of Quillaja saponaria Molina, termed Quil-A. This led to several of the benefits of the formulation of saponins into such particles being recognized. Ambitions to bring the technology further towards a possible product, such as an adjuvant for use in animal and human vaccines, prompted increased purification and characterization of the Quil-A extract. The tools available for separation and characterization of saponins at the time, during the mid to late 1980s, were reversed-phase high-performance liquid chromatography, also termed RP-HPLC, and thin layer chromatography, also termed TLC. The number of peaks revealed by RP-HPLC were numerous, while TLC revealed a few major bands. Saponin raw material was subjected to semi-preparative high-performance liquid chromatography, also termed HPLC, separations. By screening separated saponin materials for adjuvant activity in mice it was found that the saponins of major interest were residing in one of the major TLC bands. Further separation of the saponins by HPLC and screening for adjuvant activity and structure forming ability resulted in the definition of sub-groups of saponin fractions with different and interesting features. The major findings are summarized in TABLE 1, in which present terminology and major components are included for clarity and reference is made to what currently are termed fraction A, fraction B, and fraction C of Quillaja saponaria Molina saponins.

TABLE 1
Initial characterization of potentially useful fractions
of saponin materials obtained from Quil-A.

| | Adjuvant | | | | |
|---|---|---|---|---|---|
| | activity | Matrix | | Mw of | |
| | (non- | structure | | major | |
| TLC | formulated | forming | Hemolytic | known | Present |
| band | saponin) | ability | activity | component(s) | terminology |
| B1 | Poor | Poor | Low | NA | NA |
| B2 | Potent | Good | Medium | 1988 | Fraction C |
| B3 | Potent | Atypical | High | 2150 (2175 | Fraction B |
| | | structures | | and 2019) | |
| B4 | Poor/none | Good | Low | 1862 | Fraction A |

Over the years the process for fractionation of saponin raw material into fraction A and fraction C has been developed and scaled-up. For example, as described in Cox et al., PCT/AU1995/000670 (WO 96/11711), fractions A, B, and C can be prepared from the lipophilic fraction obtained on chromatographic separation of the crude aqueous Quillaja Saponaria Molina extract on a SEP-PAK column and elution with 70% acetonitrile in water to recover the lipophilic fraction. This lipophilic fraction can then be separated by semipreparative HPLC with elution using a gradient of from 25% to 60% acetonitrile in acidic water. Fraction A is the fraction that is eluted at approximately 39% acetonitrile. Fraction C is the fraction that is eluted at approximately 49% acetonitrile.

In specific embodiments, Saponin Fraction C has a purity of at least 80%, as determined by HPLC. In specific embodiments, a defined ratio of specific subcomponents within Fraction A or C are identified by mass spectrometry. In specific embodiments, cholesterol is derived from a plant-based source (phytosterol). In specific embodiments, phosphatidylcholine comprises POPC fatty acid chains. In specific embodiments, Mega-10 detergent is used at a concentration of 1% (w/v) during particle formation. In specific embodiments, Matrix-A and Matrix-C particles have a mean diameter between 30 nm and 50 nm, as measured by dynamic light scattering (DLS). In specific embodiments, Matrix-A and Matrix-C particles have a polydispersity index (PDI) less than 0.2. In specific embodiments, Matrix-A and Matrix-C particles exhibit a zeta potential between −20 mV and −40 mV. In specific embodiments, the cage-like structure is confirmed by transmission electron microscopy (TEM).

In specific embodiments, a Matrix-A to Matrix-C weight ratio is between 80:20 and 90:10. In some embodiments the Matrix-A to Matrix-C ratio is around 85:15. In specific embodiments, diafiltration uses a membrane with a molecular weight cut-off (MWCO) of 10 kDa to remove Mega-10. In specific embodiments, diafiltration is performed for a minimum of 10 cycles. In specific embodiments, a phosphate buffer solution has a phosphate concentration between 5 mM and 15 mM, pH 7.2. In specific embodiments, a temperature between 20° C. and 25° C. is maintained during the mixing and diafiltration steps. In specific embodiments, the Matrix-M adjuvant is sterilized by sterile filtration using a 0.22 μm filter.

In embodiments, a 0.5 mL dose of pneumococcal conjugate vaccine that contains Matrix-M contains between 5 μg and 100 μg of Matrix-M, more preferably between 20 μg and 80 μg of Matrix-M and more preferably between 35 μg and 65 μg of Matrix-M. In some embodiments, a 0.5 mL dose of pneumococcal conjugate vaccine that contains Matrix-M includes about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, or about 70 μg of Matrix-M.

### Formulation

The disclosure provides vaccine compositions comprising pneumococcal conjugate vaccines. In some embodiments, the disclosure provides for a pharmaceutical pack or kit comprising one or more containers filled with one or more of the components of the vaccine compositions.

Vaccine compositions disclosed herein may be used either prophylactically or therapeutically, but will typically be prophylactic. Accordingly, the disclosure includes methods for treating or preventing infection. The methods involve administering to the subject a therapeutic or prophylactic amount of the immunogenic compositions of the disclosure. Preferably, the pharmaceutical composition is a vaccine composition that provides a protective effect. In other aspects, the protective effect may include amelioration of a symptom associated with infection in a percentage of the exposed population. Vaccine compositions disclosed herein may prevent community acquired pneumonia and meningitis. Vaccine compositions disclosed herein may prevent or reduce one or more other pneumococcal bacterial diseases including, but restricted to, bronchitis, rhinitis, acute sinusitis, otitis media, conjunctivitis, sepsis, osteomyelitis, septic arthritis, endocarditis, peritonitis, pericarditis, cellulitis, and brain abscess.

Embodiments of the invention include pneumococcal conjugate vaccines that include polysaccharides from one or more pneumococcal serotypes. In some embodiments, the polysaccharide serotypes present in the vaccine include one or more serotypes selected from 1, 3, 4, 5, 6A, 6B, 7F, 8, 9N, 9V, 10A, 11A, 12F, 14, 15A, 15B, 15C, 16F, 17F, 18C, 19A, 19F, 20A, 22F, 23A, 23B, 23F, 24F, 31, 33F, and 35B. In some embodiments the polysaccharide serotypes in the vaccine include one or more serotypes selected from 3, 6A, 14 and 23F. In some embodiments the polysaccharide serotypes in the vaccine include two or more serotypes selected from 3, 6A, 14 and 23F. In some embodiments the polysaccharide serotypes in the vaccine include three or more serotypes selected from 3, 6A, 14 and 23F. In some embodiments the polysaccharide serotypes in the vaccine include the following four serotypes: 3, 6A, 14 and 23F.

The amount of each polysaccharide serotype in a human vaccine dose (a vaccine dose formulated for administration to a human) may range from 0.5 μg to 10 μg. In other words, in a vaccine containing multiple polysaccharide serotypes, at least one of the serotypes is present in an amount in the range 0.5 μg to 10 μg. In some embodiments, in a vaccine containing multiple polysaccharide serotypes, each serotype is present in an amount in the range 0.5 μg to 10 μg. In other embodiments, in a vaccine containing multiple polysaccharide serotypes, at least one of the serotypes is present in an amount in the range 1 μg to 5 μg. In some embodiments, in a vaccine dose containing multiple polysaccharide serotypes, each serotype is present in an amount in the range 1 μg to 5 μg. In other embodiments, in a vaccine containing multiple polysaccharide serotypes, at least one of the serotypes is present in an amount in the range 2 μg to 5 μg. In some embodiments, in a vaccine containing multiple polysaccharide serotypes, each serotype is present in an amount in the range 2 μg to 5 μg.

The carrier protein in a human dose of pneumococcal conjugate vaccine may be CRM197. The amount of carrier protein in a human dose of pneumococcal conjugate vaccine may be in the range from 10 μg to 100 μg. In some embodiments, the amount of carrier protein in the human dose of pneumococcal conjugate vaccine may be in the range from 20 μg to 80 μg. In some embodiments, the amount of carrier protein in the human dose of pneumococcal conjugate vaccine may be in the range from 30 μg to 65 μg.

A pneumococcal conjugate vaccine formulated for human administration may include sodium chloride in an amount of between 2-10 μg in a dose. In some embodiments, sodium chloride is present in an amount of between 2-10 μg in a dose in a 0.5 mL dose, or equivalent concentration if the dose volume is different. In some embodiments, sodium chloride may be present in a dose in an amount between 4 μg and 5 μg. In some embodiments, sodium chloride may be present in a dose in an amount between 4 μg and 5 μg in a 0.5 mL dose, or equivalent concentration if the dose is different.

One or more additional buffers may be present in a human pneumococcal conjugate vaccine dose in an amount sufficient to provide a desired buffering function. The buffer may be any pharmaceutically acceptable buffer, such as a phosphate buffer, succinate or L-histidine.

A human pneumococcal conjugate vaccine dose may include one or more pharmaceutically acceptable emulsifiers, for example polysorbate (PS) 20, PS40, PS60, PS65 or PS80, in an amount sufficient to provide a desired emulsifying function. For example, an emulsifier may be present in a 0.5 mL dose in an amount in the range between 10 μg and 10 mg, more preferably between 100 μg and 1 mg.

A human pneumococcal conjugate vaccine dose may include water as the solvent, or another pharmaceutically acceptable solvent, for the other ingredients.

As discussed above, commercially available pneumococcal conjugate vaccines include 2-4.4 μg of each polysaccharide serotype, and 30-65 μg CRM197 carrier protein in a 0.5 mL aqueous dose. Some commercially available pneumococcal conjugate vaccines include around 4.4-4.5 μg sodium chloride in a 0.5 mL dose. Some commercially available pneumococcal conjugate vaccines include a buffer such as succinate or L-histidine. They may also include an emulsifier such as polysorbate 20 (PS20) or polysorbate 80 (PS80). Other polysorbates such as PS40, PS60, and PS65 may also be used.

### Administration

In embodiments, the disclosure provides a method for eliciting an immune response against S. pneumoniae. The method involves administering an immunologically effective amount of a composition containing a pneumococcal conjugate antigen and a saponin-based adjuvant, such as Matrix-M to a subject.

Compositions disclosed herein may be administered via a systemic route or a mucosal route or a transdermal route or directly into a specific tissue. As used herein, the term “systemic administration” includes parenteral routes of administration. In particular, parenteral administration includes subcutaneous, intraperitoneal, intravenous, intraarterial, intramuscular, or intrasternal injection, intravenous, or kidney dialytic infusion techniques. Typically, the systemic, parenteral administration is intramuscular injection. As used herein, the term “mucosal administration” includes oral, intranasal, intravaginal, intra-rectal, intra-tracheal, intestinal and ophthalmic administration. Preferably, administration is intramuscular.

In embodiments, the composition is contained in a syringe for injection to the patient.

### Example 1: Immunogenicity of Prevnar-13 Pneumococcal Vaccine in Mice

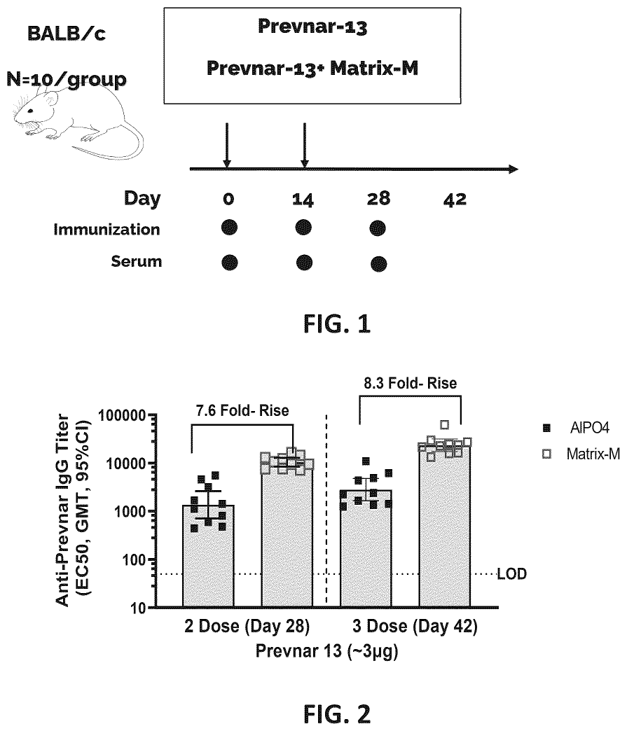

A study was performed to compare the immunological effect of Prevnar 13 with the immunological effect of Prevnar 13 supplemented with Matrix-M adjuvant, in mice. The timeline of the study protocol is shown in FIG. 1. The mice were injected with three doses of a vaccine composition, at day 0, day 14, and day 28. Blood serum was extracted from the mice for analysis on days 0, 14, 28, and 42. The mice were split into six groups of ten (N=10), and were administered different vaccine compositions as shown in the following table.

TABLE 1
Dosing Groups

| Group No. | N | Vaccine | Prevnar dose | Matrix-M dose |
|---|---|---|---|---|
| 1 | 10 | Prevnar 13 | 3 μg | None |
| 2 | 10 | Prevnar 13 | 1 μg | None |
| 3 | 10 | Prevnar 13 | 0.3 μg | None |
| 4 | 10 | Prevnar 13 | 3 μg | 5 μg |
| 5 | 10 | Prevnar 13 | 1 μg | 5 μg |
| 6 | 10 | Prevnar 13 | 0.3 μg | 5 μg |

Groups 1-3 were administered 3 μg, 1 μg, and 0.3 μg of Prevnar 13 vaccine respectively, each mouse receiving a 50 μL injection of the vaccine composition. As stated above, a 0.5 mL dose of Prevnar 13 contains approximately 2.2 μg of each of Streptococcus pneumoniae serotypes 1, 3, 4, 5, 6A, 7F, 9V, 14, 18C, 19A, 19F, 23F saccharides, and 4.4 μg of 6B saccharides, for a total of 30.8 μg of serotype polysaccharides in each dose. The doses administered to the mice were obtained by reducing the volume and concentration of pre-prepared 0.5 mL doses to produce 50 μL doses containing 3, 1, and 0.3 μg of serotype polysaccharides. Groups 4-6 were respectively administered the same vaccines as Groups 1-3, but the doses also included 5 μg of Matrix-M.

The immunological response in the mice was measured from the extracted mouse blood serum using an anti-Prevnar IgG titer and using an opsonophagocytic assay.

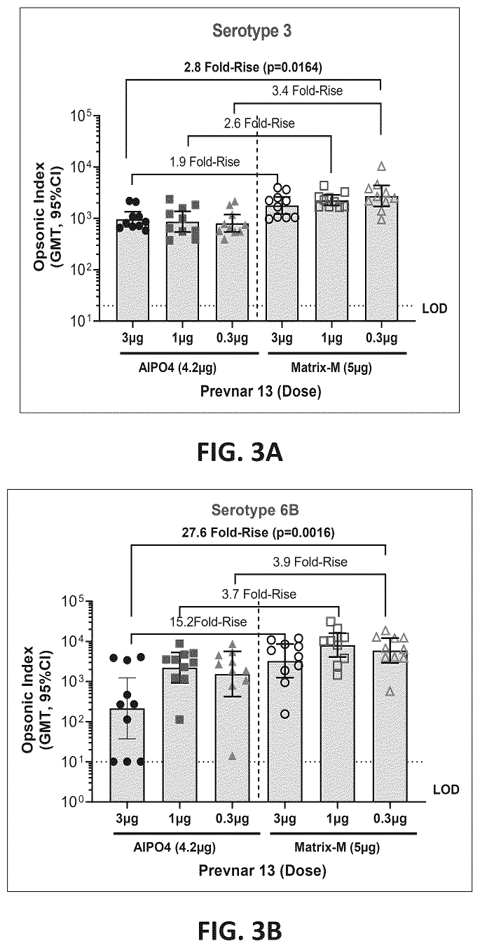

FIG. 2 shows results of an anti-Prevnar IgG titer assay for the 3 μg injections (Groups 1 and 4), from blood taken at days 28 (after 2 doses) and 42 (after 3 doses). The mice in Group 4, that received Matrix-M, showed an increased response compared to the mice in Group 1, that were administered only the Prevnar 13 composition. The results for the mice administered 2 doses of the Matrix-M-free composition (solid squares) showed an EC50 of just over 1000, while the mice that were administered 2 doses of the composition that included Matrix-M (open squares) had an average EC50 that was 7.6 fold higher. This difference between the Matrix-M and Matrix-M-free vaccines increased after the third dose, to a difference of 8.3 fold.

The multiplex opsonophagocytic assay (MOPA) was performed using the following procedure, also described in Burton et al., “Development of a fourfold multiplexed opsonophagocytosis assay for pneumococcal antibodies against additional serotypes and discovery of serological subtypes in Streptococcus pneumoniae,” Clinical and Vaccine Immunology 12 835-841 (2012). Heat-inactivated serum specimens were serially diluted in eight two-fold steps in a 96-well microtiter plate with Hanks balanced salt solution containing 0.1% gelatin (10 μL/well) and incubated with cells of the different S. pneumoniae serotypes (2,000 CFU/well) and complement (final concentration, 12.5%). The specimens were subjected to an orbital shaker for 30 min at 37° C. (final volume, 40 μL/well) (opsonization step). Differentiated HL60 cells (effector cells) were added at a 200-400:1 ratio to the target bacterium complement-serum mixture (final volume, 80 μL/well), and the mixture was incubated at 37° C. for 45 min, shaking at 250 rpm (phagocytic step). A 10 μL aliquot was removed and applied onto a 15% THY agar plate. Overlay agar containing one of four antibiotics (optichin, spectinomycin, streptomycin, and trimethoprim) and 2,3,5-triphenyltetrazolium chloride (TTC) was added, and plates were incubated overnight at 37° C. with 5% CO₂. The number of surviving colonies was enumerated, and the opsonic indices (OI) were calculated using linear interpolation. OI is defined as the reciprocal of the interpolated dilution of serum that kills 50% of bacteria.

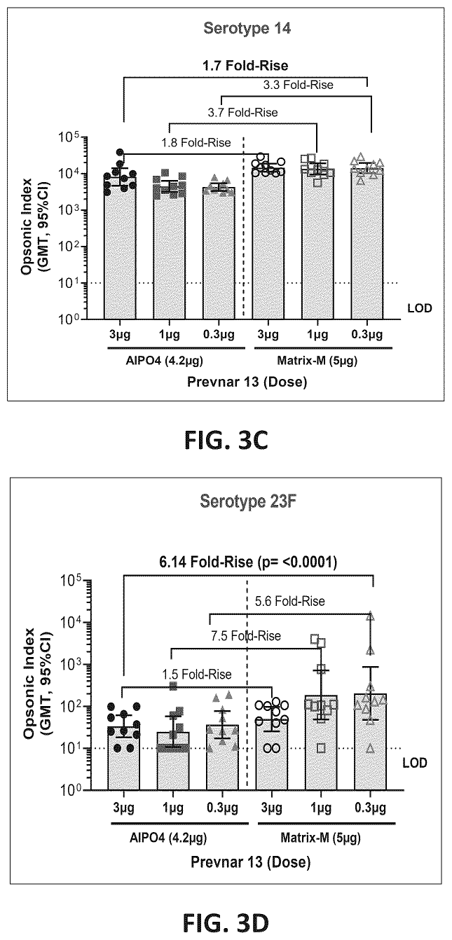

FIG. 3A shows the MOPA results for S. pneumoniae serotype 3, which is present in PCV7, Prevnar 13, Prevnar 20, Capvaxive, and Vaxneuvance. Comparing the OIs produced by the blood from each group of mice, the Matrix-M augmented vaccine (open triangles) produced a value of OI that is 3.4 fold better than for mice injected with the 0.3 μg doses of the unaugmented vaccine (solid triangles). For the mice injected with 1 μg of vaccine, the Matrix-M augmented dose (open squares) produced an OI that is 2.6 fold that resulting from the unaugmented vaccine (solid squares). For the mice injected with 3 μg of vaccine, the Matrix-M augmented dose (open circles) produced an OI almost twice that produced by the unaugmented vaccine (solid circles). Comparing the results produced by the lowest dose of the augmented vaccine (0.3 μg) with those of the highest dose of the unaugmented vaccine (3 μg), the augmented low dose was better than the unaugmented high dose vaccine by 2.8 fold.

FIG. 3B shows the MOPA results for S. pneumoniae serotype 6B, which is present in PCV7, Prevnar 13, Prevnar 20, and Vaxneuvance. Comparing the OIs produced by the blood from each group of mice, the Matrix-M augmented vaccine (open triangles) produced a value of OI that is 3.9 fold better than for mice injected with the 0.3 μg doses of the unaugmented vaccine (solid triangles). For the mice injected with 1 μg of vaccine, the Matrix-M augmented dose (open squares) produced an OI that is 3.7 fold that resulting from the unaugmented vaccine (solid squares). For the mice injected with 3 μg of vaccine, the Matrix-M augmented dose (open circles) produced an OI around 15 times that produced by the unaugmented vaccine (solid circles). Comparing the results produced by the lowest dose of the augmented vaccine (0.3 μg) with those of the highest dose of the unaugmented vaccine (3 μg), the augmented low dose was better than the unaugmented high dose vaccine by 27.6 fold.

FIG. 3C shows the MOPA results for S. pneumoniae serotype 14, which is present in PCV7, Prevnar 13, Prevnar 20, and Vaxneuvance. Comparing the OIs produced by the blood from each group of mice, the Matrix-M augmented vaccine (open triangles) produced a value of OI that is 3.3 fold better than for mice injected with the 0.3 μg doses of the unaugmented vaccine (solid triangles). For the mice injected with 1 μg of vaccine, the Matrix-M augmented dose (open squares) produced an OI 3.7 fold that resulting from the unaugmented vaccine (solid squares). For the mice injected with 3 μg of vaccine, the Matrix-M augmented dose (open circles) produced an OI 1.8 times that produced by the unaugmented vaccine (solid circles). Comparing the results produced by the lowest dose of the augmented vaccine (0.3 μg) with those of the highest dose of the unaugmented vaccine (3 μg), the augmented low dose was better than the unaugmented high dose vaccine by 1.7 fold.

FIG. 3D shows the MOPA results for S. pneumoniae serotype 23F, which is present in PCV7, Prevnar 13, Prevnar 20, and Vaxneuvance. Comparing the OIs produced by the blood from each group of mice, the Matrix-M augmented vaccine (open triangles) produced a value of OI that is 5.6 fold better than for mice injected with the 0.3 μg doses of the unaugmented vaccine (solid triangles). For the mice injected with 1 μg of vaccine, the Matrix-M augmented dose (open squares) produced an OI 7.5 fold that resulting from the unaugmented vaccine (solid squares). For the mice injected with 3 μg of vaccine, the Matrix-M augmented dose (open circles) produced an OI 1.5 times that produced by the unaugmented vaccine (solid circles). Comparing the results produced by the lowest dose of the augmented vaccine (0.3 μg) with those of the highest dose of the unaugmented vaccine (3 μg), the augmented low dose was better than the unaugmented high dose vaccine by 6.14 fold.

In all of the MOPA results described above for the different serotypes, the augmented vaccines produced an improved immunogenic response when comparing results produced using the same doses of vaccine. Furthermore, the lowest dose of the Matrix-M augmented vaccine (0.3 μg) produced results that showed an improved immunogenic response over the highest dose of the unaugmented vaccine (3 μg). This suggests that a pneumococcal conjugate vaccine that incorporates Matrix-M adjuvant may be effective at lower doses than a pneumococcal conjugate vaccine that uses only an aluminum phosphate adjuvant.

Various modifications, equivalent processes, as well as numerous structures to which the present invention may be applicable will be readily apparent to those of skill in the art to which the present invention is directed upon review of the present specification. The claims are intended to cover such modifications and devices.

As noted above, the present invention is applicable to fiber optical communication and data transmission systems. Accordingly, the present invention should not be considered limited to the particular examples described above, but rather should be understood to cover all aspects of the invention as fairly set out in the attached claims.

What is claimed is:

1. An immunogenic composition comprising:
one or more conjugated bacterial capsular polysaccharides derived from Streptococcus pneumoniae serotypes;
a saponin-based adjuvant; and
a pharmaceutically acceptable carrier.

2. The immunogenic composition of claim 1, wherein the saponin-based adjuvant comprises nanoparticles from saponins extracted from Quillaja saponaria (soapbark) trees, cholesterol, and phospholipids.

3. The immunogenic composition of claim 1, wherein the saponin-based adjuvant comprises fraction-A and fraction-C extract from Quillaja saponaria (soapbark) Molina.

4. The immunogenic composition of claim 3, wherein the saponin-based adjuvant comprises Matrix-M adjuvant comprising Fraction A Matrix and Fraction C Matrix of Quillaja saponaria Molina extract.

5. The immunogenic composition of claim 3, wherein the saponin-based adjuvant comprises Matrix-M comprising Fraction A Matrix and Fraction C matrix in a w/w ratio in the range 80:20 to 90:10.

6. The immunogenic composition of claim 5, wherein Fraction A Matrix and Fraction C Matrix are present in the saponin-based adjuvant in a w/w ratio of 85:15.

7. The immunogenic composition of claim 1, wherein the saponin-based adjuvant is present at about 5 μg to about 100 μg.

8. The immunogenic composition of claim 1, further comprising an aluminum adjuvant.

9. The immunogenic composition of claim 8, wherein the aluminum adjuvant comprises aluminum phosphate.

10. The immunogenic composition of claim 1, wherein the conjugated bacterial capsular saccharides comprise saccharides of at least one of S. pneumoniae serotype 3, serotype 6B, serotype 14 and serotype 23F.

11. The immunogenic composition of claim 10, wherein the conjugated bacterial capsular saccharides comprise saccharides of at least two of S. pneumoniae serotype 3, serotype 6B, serotype 14 and serotype 23F.

12. The immunogenic composition of claim 11, wherein the conjugated bacterial capsular saccharides comprise saccharides of each of S. pneumoniae serotype 3, serotype 6B, serotype 14 and serotype 23F.

13. The immunogenic composition of claim 1, wherein the conjugated bacterial capsular polysaccharides are present at about 0.5 μg to about 10 μg per serotype.

14. The immunogenic composition of claim 13, wherein the conjugated bacterial capsular saccharides are present at about 1 μg to about 5 μg per serotype.

15. The immunogenic composition of claim 1, wherein the pharmaceutically acceptable carrier comprises water and a buffer.

16. The immunogenic composition of claim 15, wherein the buffer comprises succinate or L-Histidine.

17. The immunogenic composition of claim 1, wherein the pharmaceutically acceptable carrier comprises an emulsifier.

18. The immunogenic composition of claim 17, wherein the emulsifier comprises at least one of polysorbate 20 (PS20), PS40, PS60, PS65, and PS80.

19. A method of preventing, treating or ameliorating a Streptococcus pneumoniae infection, disease or condition in a subject, comprising administering to the subject an effective amount of the immunogenic composition of claim 1.

20. The method of claim 19, wherein the composition is administered parenterally, transmucosally, transdermally, intramuscularly, intravenously, intradermally, intranasally, subcutaneously, or intraperitonealy.

21. The method of claim 20, wherein the composition is administered intramuscularly.

22. A prefilled syringe comprising the immunogenic composition of claim 1.
